(19) United States
(12) Patent Application Publication
(10) Pub. No.: US 2007/0019781 A1
(43) Pub. Date: Jan. 25, 2007

(54) Computer-tomographic system for carrying out a monitored intervention

(76) Inventor: Gabriel Haras, Mucke (DE)

Correspondence Address: HARNESS, DICKEY & PIERCE, P.L.C., P.O.BOX 8910, RESTON, VA, 20195, US

(21) Appl. No.: 11/491,105

(22) Filed: Jul. 24, 2006

(30) Foreign Application Priority Data
Jul. 25, 2005 (DE) 10 2005 034 684.7

(51) Int. Cl. H05G 1/60; A61B 6/00; G01N 23/00; G21K 1/12

(52) U.S. Cl. 378/4

(57) ABSTRACT

A CT system is disclosed for carrying out a monitored intervention with an instrument on a patient. The system includes an arrangement including an X-ray tube for production of a radiation beam which is moved around the patient and a detector with a large number of detector elements for measurement of the radiation intensity after passing through the patient, with the radiation beam scanning a scanning area on the patient. The system further includes an apparatus for variable orientation and positioning of the patient relative to the tube/detector arrangement, and a computation and control unit with computer programs or modules, for controlling the system and for reconstruction of the tomographic records from measurement data in the detector. A first program is stored for running and detects at least a portion of the instrument which is used for intervention, and a second program is stored for running and automatically matches the scanning area to the instrument, in accordance with presets that are provided.

### PRIORITY STATEMENT

The present application hereby claims priority under 35 U.S.C. §119 on German patent application number DE 10 2005 034 684.7 filed Jul. 25, 2005, the entire contents of which is hereby incorporated herein by reference.

### FIELD

The invention generally relates to a computer-tomographic system. For example, it may relate to one for carrying out a monitored intervention, with an instrument on a patient having at least one X-ray tube for production of a radiation beam which is moved around the patient, and having a detector with a large number of detector elements for measurement of the radiation intensity after passing through the patient, with the radiation beam scanning a scanning area on the patient, an apparatus for variable orientation and positioning of the patient relative to the tube/detector system, and a computation and control unit with computer programs, for controlling the system and for reconstruction of the tomographic records from measurement data in the detector.

### BACKGROUND

In principle, computer-tomographic systems are known, and they are in this case frequently used for the purpose of carrying out interventions on a patient with simultaneous monitoring. Interventions such as these may, for example, relate to the removal of tissue or to the treatment of possibly unhealthy tissue. A rigid needle is generally used for this purpose.

However, it is also possible to use flexible instruments, in which case, when carrying out the intervention, the operator must take care to ensure that as far as possible no organs or other sensitive tissue are caused to suffer during the course of the intervention, or that no bones are in the path.

At the moment, this is achieved by manually adjusting the scanning area of a computer-tomographic system such that it is located in the area of the actual intervention, in which case the patient table is generally moved for this purpose directly manually or by moving the table backwards and forwards in a controlled manner. With the known patient tables for a CT, this control of the patient table is carried out in a relatively uncomfortable form and results in relatively major problems for the operator, particularly if he is working with sterile hands. In addition, one particular problem occurs when the instrument must be inserted obliquely into the body of the patient, for anatomical reasons. In this case, it is particularly difficult to adjust the scanning area to the actual area of the intervention.

### SUMMARY

In at least one embodiment, a computer-tomographic system is for carrying out a monitored intervention with an instrument on a patient, which system improves the match between the location of the intervention on the patient and the scanning area of the computer-tomographic system.

The inventor, in at least one embodiment, has identified the fact that it is possible to use the automatic detection of an object in computer-tomographic records, which is intrinsically available, in such a way that the computer automatically detects an object from a specific material or an object with an already known shape, and, with the knowledge of the position of the instrument being used for the intervention, moves the scanning area relative to the patient such that it is optimally matched automatically to the position of the instrument, by way of an appropriate program, in accordance with the already defined presets.

With at least one embodiment of the invention, the operator can now actually concentrate on the actual intervention and is in each case provided with a better overview of the region of the actual intervention corresponding to the automatic adaptation of the scanning area, so that it is easier to guide the instrument that is used for the intervention without any errors. This also compensates for possible movement of the patient as a result of breathing or as a result of an active patient reaction, without the operator having to actively intervene.

On the basis of this fundamental idea, in at least one embodiment, the inventor proposes a computer-tomographic system for carrying out a monitored intervention, with an instrument on a patient which has at least one X-ray tube for production of a radiation beam which is moved around the patient, and having a detector with a large number of detector elements for measurement of the radiation intensity after passing through the patient, with the radiation beam scanning a scanning area on the patient. The system also has an apparatus for variable orientation and positioning of the patient relative to the tube/detector system, and a computation and control unit with computer programs, for controlling the system and for reconstruction of the tomographic records from measurement data in the detector. The computer-tomographic system is improved by the provision of a program which detects at least a portion of the instrument which is used for intervention, and by also providing a program which automatically matches the scanning area to the detected instrument in accordance with presets that are provided.

The apparatus for variable orientation and positioning of the patient relative to the tube/detector system may be designed in various ways. A movable patient table or a movable gantry may be used for movement in the direction of the system axis. With respect to the orientation of the longitudinal axis of the patient, the patient table can be tilted at right angles to the system axis, if required also with an additional rotational movement of the tilting axis about the system axis. On the other hand, the corresponding relative movement can also be carried out by the tube/detector system.

In one advantageous embodiment, no additional particular position identification apparatuses are required and, instead, the position of at least a portion of the instrument can be detected with the aid of CT images or directly by the change in the detector output data. As an alternative to this, however, it is also possible to provide the instrument with appropriate position sensors, so that their positions are determined for example by means of radio waves or by means of direct optical perception.

In accordance with at least one embodiment of the invention, the patient table and the X-ray tube/detector system can now be positioned relative to one another such that the scanning area overlaps the area in which the instrument is located, in accordance with a previously entered preset. In this case, by way of example, it is also possible to adjust the system such that the majority of the scanning area is located in the target area of the instrument, while areas which the instrument has also penetrated can remain unobserved.

If very broad detectors with a large number of detector rows are used for the computer-tomographic system, then the scanning area of the CT system can also be matched to the position of the instrument by movement of collimators in the tube/detector arrangement without any relative movement of the patient table and of the X-ray tube/detector system necessarily being required for this purpose. This is advantageous because no movement takes place at the patient and/or the visible system parts of the CT, and the operator is not irritated by such movement. Furthermore, the constriction of the scanning area and, if required, movement of the scanning area results in a dosage reduction in comparison to a record with the maximum possible scanning area of a broad detector.

In addition to position determination, it is also possible to detect the position of at least a portion of the instrument, thus allowing improved matching of the scanning area to the current circumstances and to the operator's wishes. For example, the position of the patient table can be varied in such a manner that the position of the scanning area is matched to the position of the instrument in accordance with a preset. In a corresponding manner, it is also possible to vary the inclination of the tube/detector arrangement instead of or in addition to the change in the position of the patient table, or else it is possible to adapt the inclination of the respectively reconstructed layers with an appropriate broad detector.

Another advantageous action is for the reconstructed CT images to be displayed at least partially obliquely, so that the operator can more easily identify the three-dimensional situation. It is likewise advantageous for the instrument to be displayed in an emphasized form on the CT display, in which case it is particularly advantageous for the emphasis to be produced by displaying the instrument using a different color.

In order to provide the operator with a better overview, the CT images can be displayed both in the scanning direction as well as axially with respect to the patient longitudinal axis. Furthermore, slice planes of the displayed CT images and/or of CT images which can be displayed can be displayed in at least one overview display of the patient. This considerably simplifies the orientation.

In a further example embodiment of the computer-tomographic system according to the invention, the inventor proposes that the intended forward feed distance of the instrument in the tissue be calculated on the basis of the already detected position of the instrument and if required also be displayed on the CT display. In this case, a preferred forward feed distance of the instrument in the tissue can be calculated and displayed taking into account automatic tissue identification and the tissue to be bypassed and/or the tissue which cannot be penetrated. Typical absorption values of the tissue, for example of bones or of specific organs, can be used for this purpose in order to provide the operator with proposals as to how the instrument can be moved further onwards. These proposals can also take account of possible bending changes of the instrument of which the computer-tomographic system is already aware.

Furthermore, it is possible for the system to have a program which emits a preferably acoustic and/or visual warning on reaching a predetermined safety distance from previously defined tissue, so that the operator is warned before possible injury, for example to organs or to major nerve systems which are located in the area of the forward feed movement.

In a further example embodiment of the computer-tomographic system, a target region can also be defined for the intervention, with the computer-tomographic system using a movement calculation program to display the optimum distance which penetrates only non-critical tissue, which movement the operator should follow with his instrument. In this case as well, it is possible to take account of the possible bending of the instrument or of any other possible change in the shape of the instrument.

Furthermore, the CT system can be equipped so that an optimum angle for the intervention relating to a target region is displayed on a display to the operator, in which case the direction of any required position change and/or orientation change of the instrument in order to achieve an optimum intervention movement with respect to a predetermined target region can also be displayed on a display to the operator.

Furthermore, the display of the required position change and/or orientation change of the instrument in order to achieve an optimum intervention movement with respect to a predetermined target region can also be displayed by way of at least one pictogram. No separate orientation of the operator or transfer of displayed images to the real situation is required for this purpose, and the direction instructions can instead of this simply be followed on the screen. It is also possible to output such instructions acoustically, in a similar manner to a vehicle navigation system. This prevents the operator from being distracted by the fact that it would otherwise be necessary to look at the display.

If, by way of example, the intervention instrument is being used to treat a tumor, then a target region can be defined and all of the areas which are located at a specific distance from a predetermined portion of the instrument and have been reached are displayed in an optically marked form, so that the operator is provided with a visual display in the form of a continuous overview of areas which have already been treated.

In addition, it is also possible for all of the displays of the scanning area to supplement the current scanning area by means of previously reconstructed volume data or CT images outside the current scanning area, thus allowing easier orientation for the operator.

For the purposes of embodiments of the invention, the method features described above may be mapped in the CT system by programs or program modules, may be stored in the data memories and program routine memories of the control and computation unit, and may be called up and processed by process units as required.

In addition, it should be noted that the described CT system and the software used in it may relate not only to C-arc machines, but also to conventional CT machines with a gantry which rotates through 360°.

### BRIEF DESCRIPTION OF THE DRAWINGS

The invention will be explained in greater detail in the following text with reference to the example embodiments and with the aid of the figures, in which only those features which are required for understanding the invention are illustrated, and in which the following reference signs are used: 1: CT system; 2: X-ray tube; 2.1: focus; 3: detector; 3.1 to 3.n detector elements; 4: system axis; 5: gantry housing; 6: movable patient couch; 7: patient; 8: opening in the gantry housing; 9: computation and control unit; 10: data and control line; 11: instrument; 12: radiation beam; 13.1 to 13.n: reconstruction planes; 14.1 to 14.n axial display planes; 15, 16 and 17: image element; 15.1, 16.1 and 17.1: overview display; 15.2, 16.2 and 17.2: detailed display of a slice; 18: collimator; 19.1 to 19.3: intervention axes; 20.1 to 20.2: forbidden regions; 21: pictogram; 22: target region; 23: image element; 100 to 115: steps in the flowchart as follows: 100: scan topo; 101: scan plan; 102: mark start/target; 103 path analysis; 104: decision point; 105: visualize optimized path; 106: match start point; 107: match the table position, gantry inclination, collimation and reconstruction to the plan; 108: intervention scan; 109: detect intervention axis; 110: analyze path; 111: decision point; 112: warning/correction proposal; 113: match the table position, gantry inclination, collimation and reconstruction to the intervention axis; 114: decision point; 115: target; Prg₁ to Prgₙ: programs; x: x-axis/tilt axis of the gantry; α: tilt angle of the gantry; z: rotation axis of the detector and X-ray tube.

In detail:

FIG. 1 shows a computer-tomographic system,

FIG. 2 shows a longitudinal section through a CT system with an inclined X-ray tube/detector arrangement,

FIG. 3 shows an example of a display of the situation from FIG. 2,

FIG. 4 shows a longitudinal slice through a scan of a patient with an inclined X-ray tube/detector system and asymmetric collimators at the start of an intervention,

FIG. 5 shows an illustration corresponding to FIG. 4, but at the end of an intervention with a reduced scanning area,

FIG. 6 shows an overview illustration of a patient in the form of a longitudinal section with a target region for an intervention and regions to be avoided during an intervention,

FIG. 7. shows a display of various slices with an instruction relating to the intervention direction, and

FIG. 8 shows a flowchart for carrying out a CT-assisted intervention.

### DETAILED DESCRIPTION OF THE EXAMPLE EMBODIMENTS

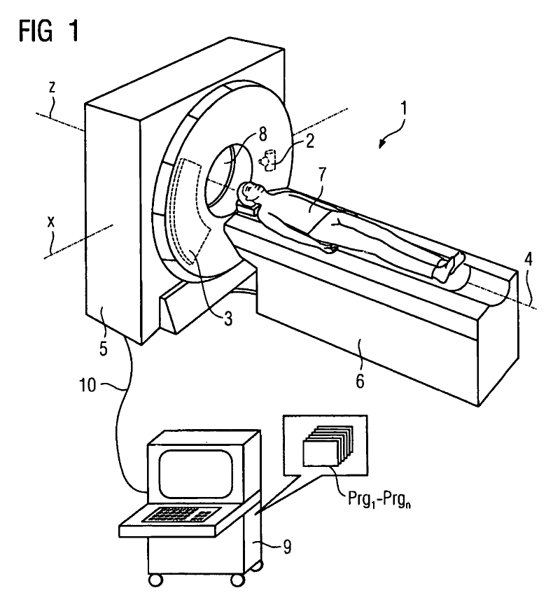

FIG. 1 shows a three-dimensional illustration of a CT system 1 according to an embodiment of the invention with a gantry housing 5 in which the gantry, which is not illustrated in any more detail, is located. An X-ray tube 2 which rotates about a system axis 4, and a detector 3 opposite it, are mounted on the gantry. A patient is located on a patient couch 6, which can be moved in a system axial direction 4, and can be moved into the beam path through an opening 8 in the gantry for scanning, where the actual intervention takes place. In addition, a tilt axis x is shown, which is arranged at right angles to the system axis z and about which the gantry can be tilted, thus allowing an oblique beam path, as is required by the invention. In this context, it should be noted that it is also within the scope of the invention for only the collimator shutter to be shifted so that only oblique beams are used for scanning, if the detector is sufficiently broad, that is to say it has a large number of rows.

The control, data gathering and data evaluation for reconstruction are carried out by the computation and control unit 9, which is connected via the data and control line 10 to the gantry and to the movable patient couch 6. Programs Prg₁ to Prgₙ are stored in this computation and control unit 9 and carry out the method according to an embodiment of the invention during operation.

It should be noted that a so-called C-arc machine can also be used as the CT system instead of the gantry with a 360° revolution as shown here, and additionally has the advantage that the accessibility to the patient is much better, thus making it easier to carry out the intervention.

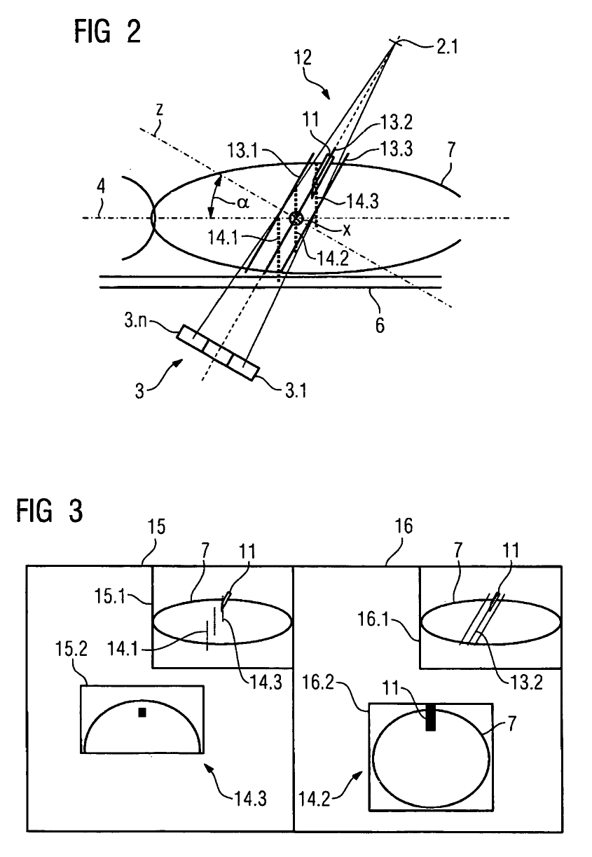

FIG. 2 shows a longitudinal section through a schematically illustrated patient 7 in the scarning area with the focus 2.1 of the X-ray tube, which emits a radiation beam 12 to an opposite detector 3 with detector elements 3.1 to 3.n. In the situation shown here, the gantry is tilted at an angle α about the x-axis, so that the system axis 4 and the rotation axis of the gantry z no longer coincide. This results in the reconstruction planes 13.1 to 13.n, which are likewise inclined through the angle α to the perpendicular. The reconstruction planes 13.1 to 13.n in the illustrated example are at the same slice angle with respect to the patient as the instrument 11 which is inserted into the patient. In order to improve the illustration and to simplify orientation for the operator, reformatted slice planes can be displayed in addition to the reconstruction planes, as indicated by 14.1 to 14.n in this case. Slice planes such as these are normally displayed axially with respect to the patient longitudinal axis, which in this case corresponds to the system axis.

FIG. 3 shows an illustration such as this of the slice planes, showing a display. This display shows two representations 15 and 16, with the representation 15 having an overview representation 15.1 in which an overview of the patient 7 is displayed and, in addition, the axially reformatted slice images 14.1 to 14.3 are displayed in the form of a longitudinal section together with the additionally illustrated intervention instrument 11.

The reformatted slice 14.3 is shown in the image part 15.2, showing a subsection of the patient 7 and the sectioned part of the instrument 11.

The image detail 16 shown on the right alongside this once again shows an overview representation 16.1 with a longitudinal section through the patient 7, in which case the three reconstructed slice planes 13.1 to 13.3 and the instrument 11 can be seen in their orientation relative to the patient in this overview. The image detail 16.2 illustrated underneath this shows—corresponding to the operator settings—the central reconstruction slice 13.2, which shows the original reconstructed image on the slice plane of the central scan with the instrument 11 which is located centrally in this scan also being illustrated here. The illustrations shown represent highly simplified illustrations of computer-tomographic slices, of course, which in reality have a much greater wealth of detail.

According to at least one embodiment of the invention, the position of the scanning area and its width are controlled by appropriate program control and previous evaluation of the detector and reconstruction data such that the optimum view in the area of the intervention being carried out is in each case available for the operator. For this purpose, an automatic calculation of the optimally required inclination angle α of the gantry relative to the patient can be taken from the available image data and this angle and the required beam width can be such that they are always up to date, thus on the one hand minimizing the dosage applied to the patient and on the other hand providing the operator with an optimum view and the best orientation capabilities.

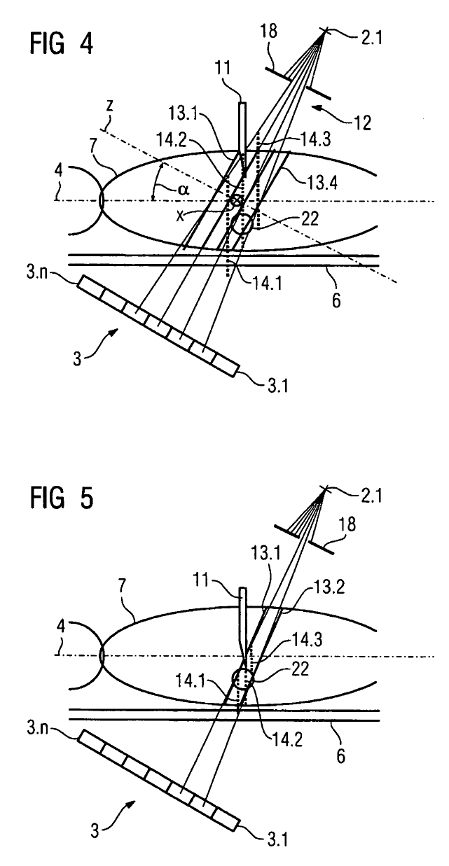

The capability for matched control of the shutter while the scanning area is at the same time set obliquely is illustrated in FIGS. 4 and 5.

FIG. 4 shows a scan at the start of the intervention, in which case a target region 22 has additionally been defined by the operator or on the basis of image identification carried out prior to this. At the start of the intervention, the collimator 18 is adjusted such that the area of the patient 7 in which the intervention is taking place is optimally illuminated. In the present example, four reconstruction planes 13.1 to 13.4 are shown. Three reformatted image planes 14.1 to 14.3 have been calculated corresponding to this up-to-date reconstructed element of the patient 7, with the central image plane 14.2 passing through the longitudinal axis of the instrument 11, and intersecting the target region 22.

As the intervention progresses, that is to say as the intervention instrument 11 penetrates deeper into the patient and approaches the target region 22, there is no longer any need to continue to scan regions which have already been penetrated, so that the beam 12 is restricted by possibly asymmetric control of the collimators 18 such that it represents precisely the critical area of the intervention instrument 11 and of the target region 22.

FIG. 5 illustrates this state by a major constriction of the radiation beam 12, so that only the planes 13.1 and 13.2 which are located in this radiation beam are reconstructed here, and only the smaller image section of this reconstruction volume is accordingly still illustrated, by way of axial reformatted slice displays 14.1 to 14.3.

It should also be noted here that, in addition to the illustrated variants, it is also possible to move the patient relative to the X-ray tube/detector system along the system axis, so that the individual beams can be tilted at an even greater angle with respect to the system axis as well by the use of edge beams of the detector.

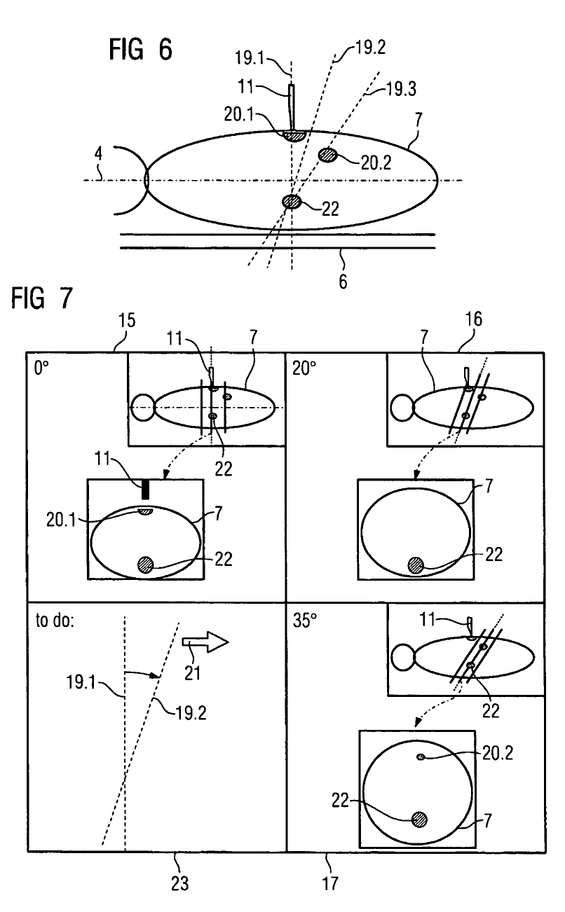

FIG. 6 shows a schematic illustration of the automated optimization of the intervention movement according to an embodiment of the invention on the patient. The illustration shows the longitudinal section through a patient as can be produced, for example, by a previous complete scan of the patient, on the one hand with the target region 22 in the patient 7 being illustrated as well as regions 20.1 and 20.2 which absolutely must not be injured during the intervention. For example, these may be organs, major blood vessels, bones or large nerve systems. In this case, the illustrated image shows a very major simplification in comparison to the actual problem.

In addition to showing the target region and the forbidden areas, intervention axes 19.1 to 19.3 are also shown, which would in principle be possible. The intervention axis 19.1 represents an axial axis, and the most direct link from the surface of the patient to the target region 22, but is tangential to the forbidden region 20.1 and for this reason cannot be used as an intervention axis. The intervention axis 19.3 which is tilted to a major extent to the right is likewise tangential to a forbidden region 20.2 on its way from the surface of the patient 7 to the target region, and thus also represents a forbidden path for the intervention. The intervention axis 19.2 represents an optimum intervention path.

On the one hand, this is the shortest intervention path from the surface of the patient 7 to the target region 22, and is not tangential to any forbidden regions on its way. An evaluation such as this can be carried out without any problems using devices/metohds available in the prior art on the basis of volume displays of a patient. This optimization of the path for the operator can be displayed in graphical form by means of appropriate image displays without placing excessive demands on the three-dimensional mental visualization capability.

It should be noted that this example, which is illustrated in a two-dimensional form here, need in no way be restricted to two dimensions but that, in the end, by way of appropriate additional details and additional displays on a second plane, the operator can also be presented with the optimum intervention channel or the intervention axis in three-dimensional space.

FIG. 7 shows an example embodiment of a display for automatic navigation for carrying out an intervention. In this case, by way of example, the display is split into four parts, and while the components 15, 16 and 17 represent overview displays and individual slice images from the angles 0°, 20°and 35°, the automatic guidance of the instrument is indicated by way of pictograms in the view element 23.

The illustration shows the situation at the start of intervention, in which case an overview scan of the patient is already available, and the target region 22 has been marked in three dimensions for intervention. The start of the intervention is governed by the position of the instrument 11. The image element 15 shows an axial illustration with a tilt angle of 0°, in which the instrument 11 is sectioned on the central reconstruction plane 13.2. In addition, the reconstruction plane 13.2 also intersects the target region 22. The illustration element 16 shows an overview of simulated reconstruction sections, as well as an intervention axis at an angle of 20°, while the image element 17 shows both the reconstruction planes and the intervention axis at an angle of 35°. As is evident from the illustrations, an intervention axis which is tilted through 20° appears to be optimum since—as can be seen from the slice images illustrated in addition to the overviews—there is no forbidden region which can be injured by the intervention here.

In a corresponding manner, the optimum intervention axis 19.2 is shown in the display part 23, in which the axial and current intervention axis 19.1 is illustrated, with a pictogram 21 illustrating the desired correction direction for the movement of the instrument 11 in order to reach the optimum intervention axis 19.2.

This display makes it possible for the operator to find the optimum intervention path without placing any excessive demands on the spatial mental visualization power.

In addition it should also be noted that the illustrated pictogram 21 can also be added to or replaced, for example, by an acoustic indication or by an appropriate speech message.

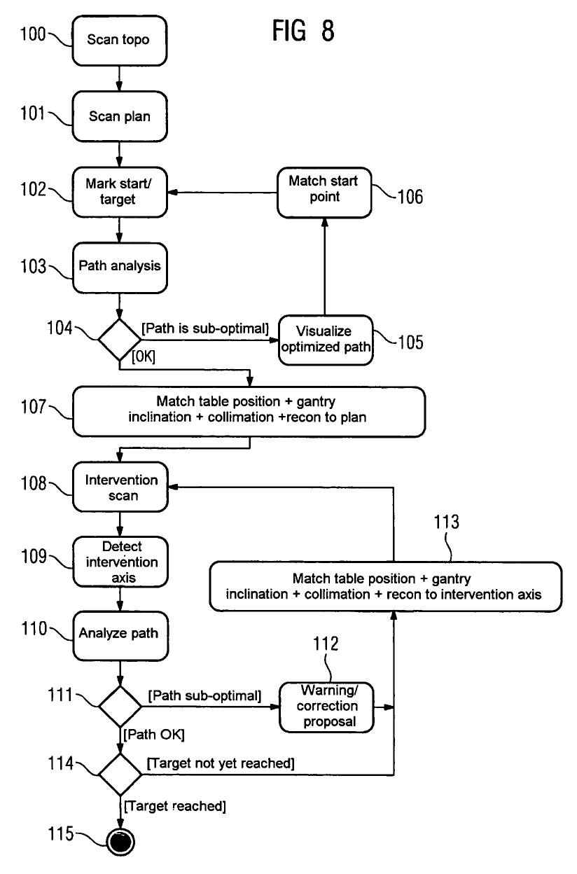

Finally, FIG. 8 shows an example of a flowchart for carrying out the intervention process according to an embodiment of the invention with the aid of the computer-tomographic system, with the steps 100 to 115. A topogram is accordingly recorded first of all in the first step 100, followed by a scan plan then being produced in the step 101, in which an overview scan of the patient is produced. In this overview scan, 102 marks the start and target of the intervention and a path analysis is carried out in step 103. If the path has been selected only sub-optimally, an optimization of the path is shown at the decision point 104 in step 105, and the start point is appropriately adapted in 106. The process then continues in 102 using this new start point. If the intervention path—on the basis of the path analysis 103—is optimal then the actual intervention is initiated at the decision point 104, with the table position, the gantry inclination, the collimation and the reconstruction path being matched to the plan in the step 107.

This is followed by an intervention scan in step 108, in which the intervention axis is detected in 109 and the corresponding path is analyzed in 110. If it is found at the decision point 111 that the path is sub-optimal, a warning is output, and/or a correction proposal is displayed in step 112, in response to which the table position, the gantry inclination, the collimation about the reconstruction plane are once again matched to the intervention axis in step 113, and the process returns to step 108. If the path is OK at the decision point 111, that is to say there are no forbidden zones in the area of the path, then step 114 detects whether the target has been reached. If this is not the case, the process jumps to step 113, otherwise the target is detected as having been reached, and the end of the intervention is displayed in step 115.

It is self-evident that the features of the invention mentioned above can be used not only in the respectively stated combination but also in other combinations or on their own without departing from the scope of the invention.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-tomographic system for carrying out a monitored intervention, with an instrument, on a patient, the system comprising:
an arrangement including an X-ray tube to produce a radiation beam to be moved around the patient, and a detector with a large number of detector elements to measure radiation intensity after passing through the patient, with the radiation beam scanning a scanning area on the patient;
an apparatus to variably orient and position the patient relative to the tube and detector arrangement; and
a computation and control unit with computer programs, to control the system and to reconstruct the tomographic records from measurement data in the detector, wherein
at least one of a first program and program module is stored to, when run, detect at least a portion of the instrument used for intervention, and
a second program is stored to, when run, automatically match the scanning area to the instrument, in accordance with presets that are provided.

2. The CT system as claimed in claim 1, wherein at least one of a further program and program module is stored by which, when run, the position of at least a portion of the instrument is detected with the aid of CT images.

3. The CT system as claimed in claim 1, wherein at least one of a further program and program module is stored by which, when run, the position of at least a portion of the instrument is detected by way of detector output data.

4. The CT system as claimed in claim 2, wherein at least one of a further program and program module is stored by which, when run, a patient table and the X-ray tube and detector arrangement are positioned relative to one another such that the scanning area overlaps the area in which the instrument is located, in accordance with the preset.

5. The CT system as claimed in claim 2, wherein at least one of a further program and program module is stored by which, when run, the scanning area of the CT system is matched to the position of the instrument by movement of collimators in the tube and detector arrangement.

6. The CT system as claimed in claim 1, wherein at least one of a further program and program module is stored by which, when run, the position of at least a portion of the instrument is detected.

7. The CT system as claimed in claim 6, wherein at least one of a further program and program module is stored by which, when run, the position of the patient table is varied in such a manner that the position of the scanning area is matched to the position of the instrument in accordance with the preset.

8. The CT system as claimed in claim-6, wherein at least one of a further program and program module is stored by which, when run, the inclination of the tube and detector arrangement is varied in such a manner that the position of the scanning area relative to the patient is matched to the position of the instrument in accordance with the preset.

9. The CT system as claimed in claim 6, wherein at least one of a further program and program module is stored by which, when run, the inclination of the reconstructed layers is varied in such a manner that their position is matched to the position of the instrument in accordance with the preset.

10. The CT system as claimed in claim 1, wherein at least one of a further program and program module is stored by which, when run, the reconstructed CT images are displayed at least partially obliquely.

11. The CT system as claimed in claim 1, wherein at least one of a further program and program module is stored by which, when run, the CT images are displayed both in the scanning direction and at right angles to the patient longitudinal axis.

12. The CT system as claimed in claim 1, wherein at least one of a further program and program module is stored by which, when run, slice planes of at least one of the displayed CT images and displayable CT images are displayed in at least one overview display of the patient.

13. The CT system as claimed in claim 1, wherein at least one of a further program and program module is stored by which, when run, the instrument is emphasized on the CT display.

14. The CT system as claimed in claim 13, wherein at least one of a further program and program module is stored by which, when run, the emphasis is produced by displaying the instrument using a different color.

15. The CT system as claimed in claim 1, wherein at least one of a further program and program module is stored by which, when run, the intended forward feed movement of the instrument in the tissue is calculated on the basis of the detected position.

16. The CT system as claimed in claim 15, wherein at least one of a further program and program module is stored by which, when run, the calculated intended forward feed distance is displayed on the CT display.

17. The CT system as claimed in claim 15, wherein at least one of a further program and program module is stored by which, when run, a preferred forward feed distance of the instrument in the tissue is calculated and displayed taking into account automatic tissue identification and at least one of the tissue to be bypassed and the tissue which cannot be penetrated.

18. The CT system as claimed in claim 15, wherein at least one of a further program and program module is stored by which, when run, takes possible bending changes of the instrument into account in the calculation of the forward feed distance.

19. The CT system as claimed in claim 15, wherein at least one of a further program and program module is stored by which, when run, at least one of a acoustic and visual warning is emitted on reaching a predetermined safety distance from previously defined tissue.

20. The CT system as claimed in claim 15, wherein at least one of a further program and program module is stored by which, when run, a target region is definable and the optimum distance, which penetrates only non-critical tissue, is displayed.

21. The CT system as claimed in claim 1, wherein at least one of a further program and program module is stored by which, when run, an optimum angle for the intervention relating to a target region is displayed on a display to the operator.

22. The CT system as claimed in claim 21, wherein at least one of a further program and program module is stored by which, when run, the direction of at least one of a required position change and orientation change of the instrument in order to achieve an optimum intervention movement with respect to a predetermined target region is displayed on a display to the operator.

23. The CT system as claimed in claim 22, wherein at least one of a further program and program module is stored by which, when run, the display of at least one of the required position change and orientation change of the instrument in order to achieve an optimum intervention movement with respect to a predetermined target region is displayed by way of at least one pictogram.

24. The CT system as claimed in claim 15, wherein at least one of a further program and program module is stored by which, when run, a target region is definable and all of the areas which are located at a specific distance from a predetermined portion of the instrument and have been reached are displayed in an optically marked form.

25. The CT system as claimed in claim 1, wherein at least one of a further program and program module is stored by which, when run, a combination of the current scanning area and the CT images reconstructed there is displayed with previously reconstructed volume data.

26. The CT system as claimed in claim 1, wherein the CT system is a C-arc machine.

27. The CT system as claimed in claim 1, wherein the CT system is a gantry with an X-ray tube which revolves through 360° around the system axis.

28. The CT system as claimed in claim 3, wherein at least one of a further program and program module is stored by which, when run, a patient table and the X-ray tube and detector are positioned relative to one another such that the scanning area overlaps the area in which the instrument is located, in accordance with the preset.

29. The CT system as claimed in claim 16, wherein at least one of a further program and program module is stored by which, when run, a preferred forward feed distance of the instrument in the tissue is calculated and displayed taking into account automatic tissue identification and at least one of the tissue to be bypassed and the tissue which cannot be penetrated.
